(19) United States
(12) Patent Application Publication
(10) Pub. No.: US 2014/0268059 A1
(43) Pub. Date: Sep. 18, 2014

(54) TRIAL FRAME

(71) Applicant: Yasutoshi Hosoki, Tokyo (JP)

(72) Inventor: Yasutoshi Hosoki, Tokyo (JP)

(21) Appl. No.: 14/207,906

(22) Filed: Mar. 13, 2014

(30) Foreign Application Priority Data
Mar. 15, 2013 (JP) 2013-054050

(51) Int. Cl. A61B 3/04

(52) U.S. Cl. CPC A61B 3/04
USPC 351/230

(57) ABSTRACT

A trial frame is provided in which a trial lens can be moved close to, or moved away from the front of the patient's face, and includes: a main bar, one end of which is connected to a hinge, and another end is turnable forward and backward relative to a patient's face; a slide member that is slidably mounted on the main bar; a sub-bar that is provided at the lower part of the slide member to be at a right angle to the main bar; a support shaft and a support plate that are slidably hung from the sub-bar; a vertical shaft that is provided on the support plate; an angle-adjusting member, one end of which is supported by the support plate to rotate around the vertical shaft; and trial lenses, each of which is hung by a fork-shaped arm at the lower part of the angle-adjusting member.

### TECHNICAL FIELD

The present invention relates to a trial frame. In particular, the present invention refers to a trial frame in which one or two trial lenses, as appropriate, that is/are mounted on the frame can be turned backward on each side of a patient's head, and can be moved close to, and away from, the front and sides of the patient's head, so that the position of the trial lenses can easily be adjusted so as to fit the variously shaped heads, especially the faces, of patients.

### BACKGROUND

A trial frame is used to hold trial lenses when an optometrist examines a patient to determine how much refractive power of lenses is to be prescribed for the patient's eyeglasses. A trial frame can also be used as a temporary frame for lenses to be temporarily worn by a patient while the patient's eyeglasses are being prepared. It is preferable that a patient's finished eyeglasses frame is properly adjusted—based on an examination using the trial frame—to appropriately fit the shape of the head, especially the face, of the patient who will wear the eyeglasses. However, conventional trial frames do not allow for trial lenses to be moved backward on each side of a patient's face, and therefore such trial frames cannot be used for preparing eyeglasses for use while participating in sports, because eyeglasses for use while participating in sports have lenses that cover not only the areas around the wearer's eyes but also some portions of each side of a wearer's face (see Patent Document 1).

Accordingly, the applicant has provided trial frames that can be used for preparing eyeglasses for use while participating in sports, as disclosed in Patent Documents 2 and 3. The trial frame disclosed in Patent Document 2 includes a horizontal supporting member that consists of a central support portion and two bars that are connected at the right and left ends, respectively, of the central support portion by a hinge. Therefore, the direction of each bar can be turned by the hinge, so that trial lenses attached to the bars can be turned backward on each side of a patient's face. That is, the angles of the trial lenses of the trial frame of Patent Document 2 can be set in the backward direction. In addition, the trial lenses are slid ably mounted on the bars via slide members, and therefore the positions of the trial lenses can be adjusted in both the left and right directions.

However, in the trial fame of Patent Document 2, if a patient whose face is relatively flat wears the trial frame, the bars may touch the left and/or right sides of the patient's head when the bars of the trial frame are turned by the hinge, as a result of which the bars cannot be turned backward fully. Accordingly, in the trial frame disclosed in Patent Document 3, the frame is provided with an angle-adjusting member that rotates around a vertical shaft in the lower part of each slide member, whereby the rotation of the angle-adjusting member enables each trial lens to be turned backward on each side of a patient's face even if the bar is turned only a small amount.

However, it is further preferable that the improved trial frame of Patent Document 3 be provided with a mechanism that moves a trial lens close to or away from a patient's face, so as to eliminate such problems as the trail lens being positioned too close to the patient's face and thereby touching the patient's eyelashes, or being positioned too far from the patient's face, even if the trial lens is turned back at an appropriate angle. These problems result because the mechanism is such that the trial lenses, being disposed on the lower side of the bars, can be slid only along the bars and turned backward on each side of a patient's face, but cannot be moved backward and forward relative to the face.

### Prior-Art Documents

### Patent Documents

Patent Document 1: JP-A-H06-123854
Patent Document 2: JP-A-2009-247775
Patent Document 3: JP-A-2012-239701

### SUMMARY

### Objective

In view of the aforesaid problems, the objective of the present invention is to provide a trial frame in which a trial lens mounted on the frame can be turned backward on each side of a patient's face, and can be moved close to, and away from, the patient's face.

### Solution to the Problem

As shown in FIG. 1, the trial frame (100) of the present invention includes: a main bar (2), one end of which is connected to a hinge (21) and the other end of which is able to move forward and backward relative to a patient's face; a slide member (4a) that is slidably mounted on the main bar (2); a sub-bar (22) that is provided on the lower part of the slide member (4a) so as to be at a right angle to the main bar (2); a support shaft (23) and a support plate (24) that slidably hang from the sub-bar (22); a vertical shaft (16) that is provided on the support plate (24); an angle-adjusting member (30), one end of which is supported by the support plate (24) so as to rotate around the vertical shaft (16); and trial lenses (9), each of which is hung by a fork-shaped arm (10) at the lower part of the angle-adjusting member (30).

The angle-adjusting member (30) is movable forward and backward relative to a patient's face.

### Advantageous Effects

The trial frame of the present invention has following advantageous features:
1. A trial lens can be brought close to, and away from a patient's face by sliding (as indicated by the reference sign h in FIG. 1) a support shaft and a support plate that hang from a sub-bar. If the lens is positioned too close to the face, to the extent that the lens touches the patient's eyelashes even if the angle of the lens is appropriate, the lens can be moved away from the face. Conversely, if the lens is positioned too far away from the face even if the angle of the lens is appropriate, the lens can be moved closer to the face.
2. Turning the lens backward on each side of a patient's head is achieved by turning a main bar and/or by rotating an angle-adjusting member. When the trial lens is angled backward, the angle of the lens is adjusted so as to cope with the various types of patients' faces, such as a face having sunken eyes, or a square-shaped face.
3. In addition to rotating the angle-adjusting member, the main bar can move forward and backward relative to the patient's face by using a hinge, which increases the adjustment range of the angle of the trial lens in the backward direction. For example, if the main bar is turned backward to the side of a patient's face and then the trial lens is rotated by the angle-adjusting member, two-stage adjustments are made, so that the trial lens is tuned backward with a large inclination angle to the side of the patient's face. Also, for example, if the main bar is turned forward from the side of a face, and then the trial lens is rotated by the angle-adjusting member, the trial lens can be tuned backward at the side of the patient's face so as to be away from the face, so as to avoid the lens touching patient's eyelashes or other part of the patient's face.

The angle-adjusting member of the trial frame can rotate both forward and backward relative to a patient's face, whereby the adjustment range of the position of the trial lens is greater than that of a trial frame without such a mechanism, wherein a main bar can be turned only backward on each side of a patient's face.

### BRIEF DESCRIPTION OF THE DRAWINGS

FIG. 1 is a perspective view of the trial frame of the present invention.

FIG. 2A is an elevation view of a hinge shown in FIG. 1.

FIG. 2B is a cross-sectional view taken from line A-A of FIG. 2A.

FIG. 3 is a perspective view showing how a trial lens is mounted on the trial frame of FIG. 1 (Example 1).

FIG. 4 is a perspective view showing the backside of a support plate shown in FIG. 3.

FIG. 5 is a perspective view showing how a trial lens is mounted on the trial frame in a different way than that shown in FIG. 1 (Example 2).

FIG. 6 is a perspective view of the support plate shown in FIG. 5.

FIG. 7 is a perspective view showing that the main bar of FIG. 1 can turn both forward and backward about the hinge and to and from the side of a patient's face.

FIG. 8A is a plan view of a trial frame worn on a patient whose face is relatively round in front.

FIG. 8B is a plan view of a trial frame worn on a patient whose face is relatively flat and square in front.

### DETAILED DESCRIPTION

Hereinafter, the trial frame of the present invention will be described with reference to the drawings.

### Examples

FIG. 1 is a perspective view of a trial frame of the present invention. The trial frame 100 includes a pair of trial lenses 9. A central support portion 1 is provided with a nose pad 8 in such a manner that the nose pad is vertically slidable. In terms of eyeglasses, the central support portion 1 is called a “bridge.” A slit is formed in the front surface of the central support portion 1, and an indicator and a scale that show at which height the nose pad 8 is positioned are provided along the slit. The nose pad 8 is fixed to the central support portion 1 by a screw. The reference sign f shows how the height of the nose pad 8 can be adjusted upward or downward.

Two main bars 2 are connected to the central support portion 1 at the right and left ends thereof via hinges 21. Each hinge 21 enables its associated main bar 2 to turn around the central support portion 1. That is, the main bar 2 can be turned by the hinge. A hinge pin 25 is inserted into the through-hole of the hinge 21. The main bar 2 can be rotated so as to adjust the backward-direction inclination angle (θ1). The degree of the backward-direction inclination angle (θ1) is shown by an angle indicator on the upper surface of the central support portion 1. The reference sign a shows how the backward-direction inclination angle (θ1) of the main bar 2 can be adjusted. Likewise, the main bar 2 can be rotated so as to adjust the forward-direction inclination angle (θ2). The reference sign b shows how the forward-direction inclination angle (θ2) of the main bar 2 can be adjusted. Also, the main bar 2, when viewed cross-sectionally, is seen to have an inverted L-shape whose upper surface is flat, so as to reduce the weight of the main bar 2, because an inverted L-shape, which only has one flange on its upper side, weighs less compared to an I-shape, which has two flanges on both its upper and lower sides. Alternatively, the main bar 2 can have a cylindrical shape. In addition, although a hinge 21 is provided at both ends of the central support portion 1, if a mechanism for height adjustment using the nose pad 8 is not necessary, the two main bars 2 can be directly connected by a hinge without the central support portion 1.

Slide members 4 (not shown in this figure) are provided on the main bar 2 for sliding the trial lens, and a slide member 4b is provided for sliding the end-piece. Sliding the slide member 4a (as indicated by the reference sign c) enables a trial lens 9 held by a fork-shaped arm 10 underside that is under the central support portion 1 to be slid to the left and right, whereby the trial lens 9 can be moved so that the patient's eye is aligned with the optical center of the trial lens 9. After the position of the trial lens 9 has been determined, the slide member 4a is fixed to the main bar 2 by a screw. The position of the slide member 4a is shown on an indicator on the upper surface of the main bar 2. Sliding the slide member 4a (as indicated by the reference sign c) enables the position of the trial lens 9 to be adjusted to the left and right. As indicated by the reference sign e, loosening or tightening screws 17 that are provided on both sides of the fork-shaped arm 10 enable the forward-inclination angle of the trial lens 9 to be adjusted. The forward-inclination angle can be read on an indicator (not shown) located between the outside lower portion of the fork-shaped arm 10 and the screw 17.

An end-piece 3 is connected to the lower part of the slide member 4b, enabling the end-piece 3 to slide horizontally. Thus, the trial frame can fit any patient regardless of the width of his/her head. After the position of the end-piece 3 has been determined, the slide member 4b is fixed to the main bar 2 by a screw. The position of the slide member 4b can be read on an indicator on the upper surface of the main bar 2. A temple 6 is mounted on one end of the end-piece 3 via a hinge that is fixed by a screw 19. The reference sign d shows how the position of the end-piece 3 can be adjusted.

The temple 6 includes a sheath 6a, so that the length of the temple can be adjusted (as indicated by the reference sign g). After the length of the temple has been determined, the temple 6 is fixed by a screw. Thus, the trial frame can fit on a patient whose ear and an eye on each side of his/her head are relatively far apart.

An earpiece 7 is rotatably mounted on the end of the sheath 6a of the temple 6. After the rotation angle has been determined, the earpiece 7 is fixed by a screw. The rotation angle can be read by the scale on an indicator. The reference sign j indicates how the rotation angle of the earpiece 7 can be adjusted.

FIG. 2A is an elevation view of the hinge shown in FIG. 1, and FIG. 2B is a cross-sectional view taken from line A-A of FIG. 2A. As shown in FIG. 2A, the hinge pin 25 is provided with serrations 27 that extend in the axial direction and that cover the entire surface of the hinge pin 25. Similar serrations are provided on a hinge hole of the central support portion 1, so that the hinge pin 25 is fixed to the central support portion 1. Also, as is shown in FIG. 2B, projections 28 are provided on the main bar 2 so that the projections 28 penetrate through the hinge hole of the central support portion 1 so as to engage with the serrations 27. A spring is incorporated into the projection 28, so that when the main bar 2 is rotated, the projection 28 is pushed upward by the crest parts of the serrations 27, and when the rotation of the main bar 2 causes the projection 28 to arrive at a trough part of a serration 27, the projection 28 is pushed downward by the spring, so that the projections 28 stably engage with the serrations 27. Thus, the backward-direction inclination angle (θ1) or the forward-direction inclination angle (θ2) can be increased in steps from pitch to pitch of the serrations 27.

FIG. 3 is a perspective view showing how the trial lens of FIG. 1 is mounted on the trial frame (Example 1). The trial lens 9 in FIG. 3 is for a patient's right eye. The slide member 4a is mounted on the main bar 2 so as to be slidable, and a sub-bar 22 is provided in the lower part of the main bar 2 in such a way that the sub-bar 22 is at a right angle to the main bar 2. A support shaft 23 is provided on the sub-bar 22 so as to be slidable horizontally, and a support plate 24 is provided at the bottom of the support shaft 23. A vertical shaft 16, which is a bolt, is provided on the support plate 24 at the position where the vertical shaft 16 is located farther from the center of the support plate 24 and nearer to the central support portion 1. A plate-like angle-adjusting member 30 is provided on the underside of the support plate 24 so as to rotate around the vertical shaft 16. The vertical shaft 16 is located farther from the center of the support plate 24 and nearer to the central support portion 1, and therefore, in the case of the lens for a patient's right eye, as shown FIG. 3, the right side of the trial lens 9 can move backward on that side of the patient's face, and the left side of the trial lens 9 does not move forward very much relative to the face. One side of the support plate 24 is folded so as to support the angle-adjusting member 30. A fork-shaped arm 10 that sandwiches a lens-holding ring 5, on which the trial lens 9 is mounted, is connected to the lower part of the angle-adjusting member 30. The reference sign h indicates how the support shaft and the support plate can slide. Thus, the trial lens 9 can be adjusted in such a way that the lens can be moved close to, or away from, the patient's face. That is, the position of the trial lens 9 can be adjusted forward and backward. The reference sign i shows how a rotation angle θ3, which will be explained below, can be adjusted.

FIG. 4 is a perspective view showing the backside of the support plate 24. Six engaging holes 13 are provided on the rear side of the support plate 24, but the number of the holes is not limited to six. The engaging holes 13 correspond to the positions of the rotation angles of 0°, 5°, 10°, 15°, 20°, and 25°, respectively. In contrast, a rotating plate 30a of the angle-adjusting member 30 is provided with an engagement ball 14. A rotation angle θ3 for the rotating plate 30a is set by rotating the angle-adjusting member 30 in such a way that the engagement ball 14 engages with an engaging hole 13, whereby the feeling of rotating the angle-adjusting member 30 can surely be transmitted to a user's fingers, so that a very small rotation angle can be set. In addition, the smallest rotation angle θ3 of the rotating plate 30a formed by one rotation is not limited to 5°, but can be much smaller. Also, the maximum rotation angle can be set at a degree larger than 25°. In this example, 0° is set so that the rotating plate 30 is aligned with the main bar 2, whereby if the angle is set to a positive (plus) number, i.e., in the direction in which the rotating plate is turned backward on that side of a patient's face, but the angle can be set to a negative (minus) number, in which the rotating plate is turned forward on that side of a patient's face.

FIG. 5 is a perspective view of Example 2, showing how a trial lens is mounted on the trial frame in a different way than that shown in FIG. 1. The trial lens 9 of FIG. 5 is for a patient's left eye. The support plate 24 of Example 2 can be narrow. A slide member 4a is slidably mounted on the main bar 2, and a sub-bar 22 is provided on the lower part of the slide member 4a so as to be at a right angle to the main bar 2. A support shaft 23 is slidably mounted on the sub-bar 22, and a support plate 24 is provided at the bottom part of the support shaft 23. A vertical shaft 16, which is a bolt, is provided on the support plate 24. An angle-adjusting member 30 is provided on the support plate 24 at the lower side thereof so as to rotate around the vertical shaft 16. The trial lens 9 can freely rotate forward or backward relative to a face. A fork-shaped arm 10 that sandwiches a lens-holding ring 5, on which the trial lens 9 is mounted, is connected to the lower part of the angle-adjusting member 30.

FIG. 6 is a perspective view of the support plate of FIG. 5. The angle-adjusting member 30, which is cylindrical-shaped, is slidably mounted on the vertical shaft 16. There is a triangular projection 18 at the upper end of the angle-adjusting member 30, and an engagement ball 14 is provided underneath the triangular projection 18. Also, the support plate 24 is provided with engaging holes 13 that set rotation angles. The engaging holes 13 correspond to the positions of the rotation angles of 0°, 5°, 10°, 15°, 20°, and 25° respectively. The rotation angle θ3 is set by holding and rotating the angle-adjusting member 30 in such a way that the engagement ball 14 engages with an engaging hole 13. In addition, the vertical shaft 16 is located away from the central axis of the trial lens 9 so that the vertical shaft 16 is positioned near the central support portion 1 (not shown). Accordingly, even if the trial lens 9 is turned backward to the near side of a patient's face, the portion of the trial lens 9 near the nose does not move forward much relative to the face.

FIG. 7 is a perspective view showing that the main bars 2 that extend to the left and right, respectively, from the central support portion of FIG. 1, are connected by hinges, and each main bar 2 can turn both forward and backward on the hinge to its respective the side of the patient's face. θ1 indicates a backward-inclination angle, and θ2 indicates a forward-inclination angle. That is, each main bar 2 can turn both backward and forward. Where the angle of the trial lens 9 in the backward direction is set to Sd, then the rotation angle of the angle-adjusting member 30 is Sd=θ1+θ3 or Sd=θ3−θ2. θ3.

FIG. 8A is a plan view of a patient whose face is relatively round in front and who is wearing a trial frame, and FIG. 8B is a plan view of a patient whose face is relatively flat and square and who is wearing a trial frame. As is shown by the drawing of the right side of the trial frame 100 in FIG. 8A, in order to turn the trial lens 9 backward at the side of a face so that the lens is angled backward while a patient wears the trial frame 100, the main bar 2 is turned at a backward-inclination angle (θ1). If the patient has sunken eyes, so that the trial lenses 9 are initially positioned away from the eyes, the sunken eyes can be coped with by sliding the support shaft 23 along the sub-bar 22 toward the face. If the angle θ is not far enough to the back to turn the trial lens 9 appropriately, a larger angle (Sd) in the backward direction can be formed by adding a rotation angle θ3 to the angle θ1 by adjusting the angle-adjusting member 30 so as to form a larger angle (Sd) (Sd=θ1+θ3).

As is shown by the drawing of the right side of the trial frame 100 in FIG. 8B, if a patient has a square-shaped face, it is not possible for the main bar 2 to form a large backward-direction inclination angle (θ1) while the patient is wearing the trial frame 100. But if a small backward-inclination angle is to be formed and the trial lens 9 touches the patient's eyelashes, the support shaft 23 can be slid along the sub-bar 22 so as to move away from the face. As is shown by the drawing of the left side of the trial frame 100 in FIG. 8B, if the main bar 2 is turned so as to form the forward-direction inclination angle (θ2) and thereby to move away from the face, and subsequently the angle-adjusting member 30 is rotated so that the rotation angle θ3 is adjusted, the trial lens 9 can be turned at a prescribed inclination angle (Sd) in the backward direction (Sd=θ03−θ2).

### INDUSTRIAL APPLICABILITY

The trial frame of the present invention can be effectively used for preparing eyeglasses for use while participating in sports.

1. A trial frame, comprising:
a main bar, one end of which is connected to a hinge, and the other end of which is able to turn forward and backward relative to a patient's face;
a slide member slidably mounted on the main bar;
a sub-bar provided at the lower part of the slide member so as to be at a right angle to the main bar;
a support shaft and a support plate slidably hung from the sub-bar;
a vertical shaft provided on the support plate;
an angle-adjusting member, one end of which is supported by the support plate to rotate around the vertical shaft; and
trial lenses, each of which is hung by a fork-shaped arm at the lower part of the angle-adjusting member.

2. The trial frame according to claim 1, wherein the angle-adjusting member can rotate forward and backward relative to a patient's face.
